(19) United States
(12) Patent Application Publication
(10) Pub. No.: US 2018/0245026 A1
(43) Pub. Date: Aug. 30, 2018

(54) SYSTEM FOR DISPENSING A SOLUTION OF NITRIC OXIDE IN DEIONIZED AND/OR DEOXYGENATED WATER FROM A VESSEL

(71) Applicants: Kori Dunn, Buffalo, NY (US); Lloyd A. Brown, Amherst, NY (US); Paul K. Oetinger, Gasport, NY (US)

(72) Inventors: Kori Dunn, Buffalo, NY (US); Lloyd A. Brown, Amherst, NY (US); Paul K. Oetinger, Gasport, NY (US)

(21) Appl. No.: 15/891,697

(22) Filed: Feb. 8, 2018

Related U.S. Application Data
(60) Provisional application No. 62/464,463, filed on Feb. 28, 2017.

(51) Int. Cl. C11D 3/48; B01D 11/02; C11D 17/00; A61L 2/00; C11D 7/10; A61L 2/18

(52) U.S. Cl. CPC C11D 3/48; B01D 11/0288; A61L 2/18; A61L 2/0088; C11D 7/105; C11D 17/0043

(57) ABSTRACT

A pressurized containment vessel for a gas/liquid mixture of nitric oxide in deionized and/or deoxygenated water and a system for dispensing the gas/liquid mixture of nitric oxide in deionized and/or deoxygenated water from the containment vessel are provided. The containment vessel and dispensing system are configured so as to maintain the gas/liquid mixture within the containment vessel at a constant pressure above ambient pressure as the gas/liquid mixture of nitric oxide in deionized and/or deoxygenated water is stored in and depleted from the containment vessel thus preventing the nitric oxide from coming out of the solution.

### CROSS REFERENCE TO RELATED APPLICATIONS

The present application claims priority from U.S. Provisional Patent Application Ser. No. 62/464,463 filed on Feb. 28, 2017; the disclosure of which is incorporated by reference herein.

### TECHNICAL FIELD

The present invention relates to a multi-chamber containment vessel and a system for dispensing a gas/liquid mixture of nitric oxide in deionized and/or deoxygenated water from the containment vessel. More particularly, the present invention relates to a system for dispensing a gas/liquid mixture of nitric oxide in deionized and/or deoxygenated water from a containment vessel that maintains the gas/liquid mixture within one of the chambers within the containment vessel at a prescribed pressure range above ambient pressure as the volume of the gas/liquid mixture in the vessel changes and prevents nitric oxide from coming out of the deionized and/or deoxygenated water in the vessel.

### BACKGROUND

There have recently been developed various systems and methods for sanitizing or disinfecting the surface of a body part or item using a gas/liquid mixture of nitric oxide in deionized and/or deoxygenated water. One example of such system and method is described in U.S. Patent Application Publication No. 2014/0186211. The challenge in using a gas/liquid mixture of nitric oxide in deionized and/or deoxygenated water in such sanitation and disinfection systems is the degradation of the solution over time as contaminants may infiltrate the solution prior to dispensing. Another problem associated with such gas/liquid mixtures of nitric oxide in deionized and/or deoxygenated water, is that the nitric oxide gas may come out of the deionized and/or deoxygenated water during storage, transport and dispensing of the gas/liquid mixture resulting in inconsistent nitric oxide concentration in the dispensed mixture.

What is needed, therefore, is a containment vessel for a gas/liquid mixture comprising nitric oxide in deionized and/or deoxygenated water configured to be operably integrated into such nitric oxide based sanitation and disinfection systems and associated methods. The containment vessel must prevent infiltration of contaminants into the gas/liquid mixture comprising nitric oxide in deionized and/or deoxygenated water and must be capable of maintaining a substantially constant nitric oxide concentration in the deionized and/or deoxygenated water as the solution is stored within the vessel and as the solution is dispensed or depleted from the vessel over time.

### SUMMARY OF THE INVENTION

The present invention may be characterized as a vessel for storage, transport and dispensing of a gas/liquid mixture comprising nitric oxide in deionized and/or deoxygenated water, the vessel comprising: (i) a housing having a body section, a dispensing end, and an opposite end, the body section defining an interior chamber having one or more interior surfaces; (ii) a moveable piston having a distal end, a proximal end, and one or more side surfaces disposed within the interior chamber such that the one or more side surfaces are sealingly engaged with the one or more interior surfaces of the interior chamber to define the first section of the interior chamber between the distal end of the piston and the dispensing end of the housing and to define the second section of the interior chamber between to the distal end of the piston and the opposite end of the housing. The dispensing end of the housing includes at least one dispensing port through which the gas/liquid mixture of nitric oxide in deionized and/or deoxygenated water exits the first section of the interior chamber as the moveable piston moves towards the dispensing end of the housing. In addition, at least one of the one or more surfaces of the moveable piston remains in contact with a liquid surface of the gas/liquid mixture of nitric oxide in deionized and/or deoxygenated water in the first section of the interior chamber to prevent nitric oxide from coming out of the deionized and/or deoxygenated water.

Alternatively, the present invention may be characterized as a dispensing system for a gas/liquid mixture of nitric oxide gas in deionized and/or deoxygenated water, the dispensing system comprising: (i) a container having a body section defining an interior chamber having one or more interior surfaces; a dispensing end; and an opposite end; (ii) a moveable piston disposed within the interior chamber of the container, the moveable piston having a distal end, a proximal end, and one or more side surfaces, the moveable piston configured such that the one or more side surfaces are sealingly engaged with the one or more interior surfaces of the interior chamber of the container to define a first section of the interior chamber between to the distal end of the moveable piston and dispensing end of the container and a second section of the interior chamber between to the proximal end of the moveable piston and the opposite end of the container; and (iii) a motive source in operative association with the container and the moveable piston and configured to cause the moveable piston to traverse the interior chamber of the container. The dispensing end of the container includes at least one port through which the gas/liquid mixture of nitric oxide in deionized and/or deoxygenated water is dispensed as the moveable piston traverses the interior chamber of the container toward the dispensing end of the container. The distal end of the moveable piston remains in contact with a liquid surface of the gas/liquid mixture of nitric oxide in deionized and/or deoxygenated water in the first section of the interior chamber so as to prevent nitric oxide from coming out of the deionized and/or deoxygenated water.

Preferably, the gas/liquid mixture of nitric oxide in deionized and/or deoxygenated water is maintained at a prescribed pressure range above ambient pressure as the volume of the gas/liquid mixture within the first section of the interior chamber changes. Also, the gas/liquid mixture of nitric oxide in deionized and/or deoxygenated water in the first section of the interior chamber is sealably isolated from any contaminants present in the second section of the interior chamber or from outside the housing.

The moveable piston preferably traverses the interior chamber toward the dispensing end in response to either a pneumatic force, a hydraulic force, or a mechanical force applied to or transferred to the moveable piston, or various combinations of a mechanical force, such as a spring, together with a pneumatic or hydraulic force. The dispensing system and containment vessel, including the moveable piston and other such components, are preferably constructed of materials selected from the group consisting of polyvinylchloride, aluminum, stainless steel, and fluoropolymer elastomers.

The dispensing system and containment vessel also may include an alarm, a gauge, an electronic display, a processor and one or more sensors configured to ascertain one or more characteristics of the gas/liquid mixture of nitric oxide in deionized and/or deoxygenated water contained in vessel. The one or more sensors may include a temperature sensor, a pressure sensor, and/or a position detection sensing arrangement to ascertain the position of the moveable piston and hence the volume of the gas/liquid mixture of nitric oxide in deionized and/or deoxygenated water contained remaining in the vessel. The volume of the gas/liquid mixture of nitric oxide in deionized and/or deoxygenated water contained in the vessel as well as the temperature and pressure are preferably indicated or displayed on the electronic display and/or gauge. A visual and/or audible alarm is also activated when the volume, temperature or pressure of the gas/liquid mixture of nitric oxide in deionized and/or deoxygenated water contained in the vessel reach prescribed thresholds or are outside permissible ranges. Alternatively, the vessel could be equipped with ports or with other means of communicating this information to an independent system (such as the delivery device) that would have these alarm and display features.

An additional feature or benefit of the present containment vessel and dispensing system is that it can also be used to ensure dispensing of the solution at the required volumetric rate for use in sanitizing or disinfecting the surface of a body part or item.

### BRIEF DESCRIPTION OF THE DRAWINGS

While the present invention concludes with claims distinctly pointing out the subject matter that Applicants regard as the invention, it is believed that the invention will be better understood when taken in connection with the accompanying drawings in which:

FIG. 1 is a perspective view of a dispensing system with a containment vessel for a gas/liquid mixture comprising nitric oxide in deionized and/or deoxygenated water in accordance with an embodiment of the present invention; and

FIG. 2 is a cross-section view of the dispensing system with a containment vessel of FIG. 1.

### DETAILED DESCRIPTION

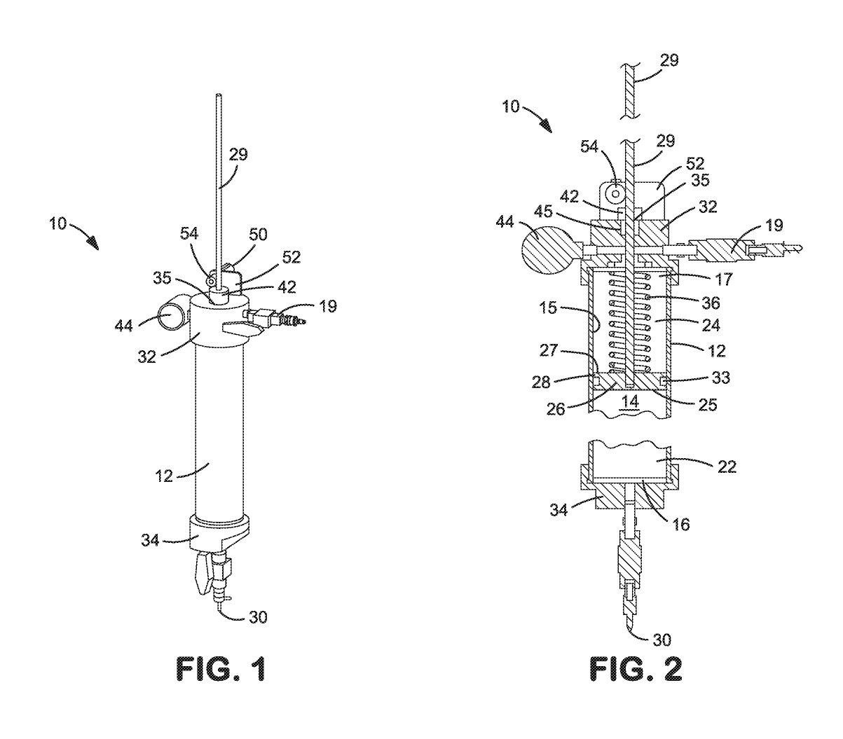

Turning now to FIGS. 1 and 2, there is shown a containment vessel 10 configured to hold a gas/liquid mixture of nitric oxide in deionized and/or deoxygenated water solution such as a gas/liquid mixture comprising nitric oxide in deionized and/or deoxygenated water. The containment vessel 10 comprises a generally cylindrical housing 12, a moveable structure disposed within the housing 12; a dispensing port 30, and one or more filling ports. In the illustrated embodiment, the housing 12 includes a body section defining an interior chamber 14 with one or more interior surfaces 15, a dispensing end 16, an opposite end 17. Interior chamber 14 is partitioned by the moveable structure into a first section 22 configured to hold the gas/liquid mixture comprising nitric oxide in deionized and/or deoxygenated water and a second section 24 that is sealably segregated from the first section 22 and does not contain or hold any portion of the gas/liquid mixture comprising nitric oxide in deionized and/or deoxygenated water.

In the illustrated embodiment of FIGS. 1 and 2, the moveable structure is a piston 25 that traverses the interior chamber 14 of the housing 12. The piston includes a distal end 26, a proximal end 27, and one or more side surfaces 28 disposed within the interior chamber such that the one or more side surfaces 28 are sealingly engaged with the one or more interior surfaces 15 of the interior chamber 14. The location and positioning of the piston 25 defines the first section 22 of the interior chamber 14 between the distal end 26 of the piston 25 and the dispensing end 16 of the housing 12 and the second section 24 of the interior chamber 14 between to the proximal end 27 of the piston 25 and the opposite end 17 of the housing 12. The actuating force to move piston 25 in a downward direction toward the dispensing end 16 may be a spring 36 (e.g. constant force spring), a pneumatic force or hydraulic force applied to the proximal end 27 of the piston that moves piston 25 such as by a bellows, or other electrical or mechanical force such as a motor (not shown) connected to the piston 25 via piston rod 29. Where a pneumatic or hydraulic force is used to move the piston 25 within the interior chamber, a pneumatic or hydraulic filling port and valve arrangement 19 is located proximate opposite end 17 of the housing 12 in fluid communication with the second section 24 of interior chamber 14.

The dispensing end 16 of the housing 12 includes at least one dispensing port 30 through which the gas/liquid mixture of nitric oxide in deionized and/or deoxygenated water exits the first section 22 of the interior chamber and is dispensed as the piston 25 moves within the interior chamber toward the dispensing end 16 of the housing 12. In addition, a filling port configured to add the gas/liquid mixture of nitric oxide in deionized and/or deoxygenated water to the first section 22 of the interior chamber is disposed on the body section 13 of the housing 12 and preferably located proximate the dispensing end 16. In some embodiments, the filling port and dispensing port may be the same.

The preferred embodiment of the containment vessel 10 is constructed from a high quality stainless steel (316SS). This common grade of stainless steel is preferred for applications of nitric oxide in in deionized and/or deoxygenated water because of its corrosion resistance properties. At the bottom of the housing 12 proximate dispensing end 16 a 316SS bottom cap 34 is disposed. This bottom cap 34 contains a connection for a standard 316SS ball valve (not shown). At the top of the housing 12 proximate the opposite end 17, a 316SS top cap 32 is disposed. The top cap 32 preferably contains an opening 35 for the piston rod 29 and connections for a pressure gauge 44 and valve arrangement 19, such as a standard 316SS ball valve. The ball valves may be attached to 316SS quick connects for ease of use. The piston 25 is also preferably a 316SS piston assembly with a connection for the piston rod 29, also made of 316SS, and a groove (not shown) for a gasket or O-ring (not shown). The gaskets and O-rings 33 are used to ensure that the piston 25 maintains pressure-tight contact with the interior surface 15 of the housing 12 and to prevent contaminants from entering from the second section 24 of the interior chamber 14 to the liquid/gas mixture within the first section 22 of the interior chamber l4. Additional gaskets (not shown) may be placed between the housing 12 and top cap 32; between the housing 12 and bottom cap 34; and between the piston rod 29 and top cap 32. A 316SS retainer 42 may also be used to hold the O-ring 45 in place between the piston rod 29 and the top cap 32 to maintain pressure-tight seals.

Alternative materials suitable for use in the construction of the containment vessel and components shown in the FIGS. 1 and 2 include any materials generally compatible with nitric oxide. Examples of such materials: include carbon steel, stainless steel, copper, aluminum, monel alloys, fluorocarbon based polymers (e.g. Kel-F, Teflon, Tefzel, Kynar) polyvinylchloride, polycarbonates, and various fluoropolymer elastomers including those sold under the trade names Kalrez and Viton. However, it should be understood that nitric oxide can be corrosive to certain metals such as aluminum, carbon steel, and low quality stainless steels when in the presence of water.

As described above, the distal end surface of the moveable piston remains in contact with the liquid surface of the gas/liquid mixture of nitric oxide in deionized and/or deoxygenated water in the first section of the interior chamber so as to prevent nitric oxide from coming out of the deionized and/or deoxygenated water. Such arrangements also maintain the gas/liquid mixture of nitric oxide in deionized and/or deoxygenated water in the first section at a prescribed pressure range above ambient pressure as the volume of the gas/liquid mixture within the first section of the interior chamber changes by virtue of the gas/liquid mixture of nitric oxide in deionized and/or deoxygenated water being dispensed from the vessel. Finally, such arrangement also sealably isolates the gas/liquid mixture of nitric oxide in deionized and/or deoxygenated water in the first section of the interior chamber from any contaminants present in the second section of the interior chamber or from outside the container or vessel.

In the embodiment of FIGS. 1 and 2, a spring 36 is used to ensure that the piston maintains contact with the surface of the gas/liquid mixture during both storage and use. In this embodiment, the length of the spring when it is fully decompressed must be greater than or equal to the length of the housing, such that the spring is capable of driving the piston all the way to the dispensing end of the housing while keeping the distal end of the piston in contact with the surface of the gas/liquid mixture and together with any pneumatic or hydraulic forces acting on the piston, keep the gas/liquid mixture within a prescribed pressure range above ambient pressure so as to prevent nitric oxide from coming out of the deionized and/or deoxygenated water.

In the embodiment of FIGS. 1 and 2, the force on the piston required to maintain the gas/liquid mixture in the first section at a prescribed pressure range above ambient pressure and to keep the distal end of the piston in contact with the surface of the gas/liquid mixture is applied by both a spring and a pneumatic/hydraulic force. When the first section of the interior chamber vessel is full of the gas/liquid mixture, i.e. during storage and transport, the spring acts to provide the requisite force on the proximal end of the piston. Then, during use, the containment vessel is connected to either a compressed gas source or hydraulic fluid source via the filling port that is in fluid communication with the second section of the interior chamber. As the gas/liquid mixture of nitric oxide in deionized and/or deoxygenated water is dispensed, the pneumatic force from the compressed gas or the hydraulic force from the fluid moves the piston in a downward direction to keep the distal end of the piston in contact with the surface of the gas/liquid mixture and keeps the gas/liquid mixture within a prescribed pressure range above ambient pressure so as to prevent nitric oxide from coming out of the deionized and/or deoxygenated water.

The use of a spring together with a compressed gas or liquid source makes sense because the energy stored in the spring can be used to maintain the gas/liquid mixture within the first section of the interior chamber within a prescribed pressure range above ambient pressure during storage and transport when no energy is available in the form of a compressed gas or liquid. Then, when the vessel is connected or inserted into a delivery apparatus, a compressed gas or liquid source is available to maintain the force on the piston constant during use.

The piston assembly proposed in the embodiment of FIGS. 1 and 2 also simplifies filling of the vessels with the gas/liquid mixture of nitric oxide in deionized and/or deoxygenated water. Since, it is important that the interior chamber of the vessel be deoxygenated before the gas/liquid mixture of nitric oxide in deionized and/or deoxygenated water is added to the interior chamber to avoid the nitric oxide reacting with oxygen to form acidic byproducts, the movement of the piston to the dispensing end of the housing aids keeping the interior chamber of the vessel generally oxygen-free.

For example, when the piston is lowered all the way to the dispensing end of the housing, the first section of the interior chamber will be completely evacuated and generally oxygen-free. The vessel can then be filled with the gas/liquid mixture of nitric oxide in deionized and/or deoxygenated water preferably from the dispensing port or alternatively from a separate filling port located proximate the dispensing end of the housing. Prior to filling the first section of the vessel with the gas/liquid mixture of nitric oxide in deionized and/or deoxygenated water, it may be beneficial to pull a vacuum or partial vacuum in the first section so as to ensure there is little or no oxygen present in the first section of the interior chamber before filling.

Knowing the location or position of the piston 25 or piston rod 29 allows one to ascertain or calculate the volume of gas/liquid mixture remaining in the containment vessel and therefore to determine when the gas/liquid mixture is depleted or how many further dispenses or doses remain in the vessel prior to replacing, refilling or changing the containment vessel. For example, the location or position of the piston 25 within the interior chamber 14 with respect to a reference point on the interior surface 15 of the interior chamber 14 can be used to determine the volume of gas/liquid mixture remaining in the containment vessel. Alternatively, the position or location of the piston rod 29 with respect to a reference point can also be used to determine the volume of solution remaining in the containment vessel. In the illustrated embodiment, the movement and location of the piston rod 29 relative to the reference point can be discerned using a shaft roller 54 and a potentiometer 50 attached to the top cap 32 using a bracket 52.

One embodiment of a dispensing system for sanitizing or disinfecting the surface of a body part or item using a gas/liquid mixture of nitric oxide in deionized and/or deoxygenated water is shown and described in U.S. Patent Application Publication No. 2014/0186211, the disclosure of which is incorporated by reference herein. Other embodiments of the gas/liquid mixture of nitric oxide in deionized and/or deoxygenated water dispensing systems contemplated for use with the presently disclosed containment vessel include stationary dispensing systems that are affixed to a wall, a table or free-standing units. Alternatively, the dispensing systems may be configured as portable dispensing systems disposed on a cart and fluidically coupled to one or more containment vessels also disposed on the cart. Such portable dispensing systems might typically include a wheeled cart that can accommodate one or more containment vessels of various sizes and types and a surface that allows for mounting of the dispensing system and accessories. The wheeled cart could readily be moved to multiple locations within a facility where such sanitation and disinfection is required.

The typical dispensing system would be configured to operate with pressurized containment vessels at pressures between about 0 psig and 1500 psig, and more preferably at pressures between about 5 psig and 100 psig. The size of the containment vessel is preferably between about 0.1 liters and 200 liters, and more preferably between about 0.5 liters and 4.0 liters. Containment vessels with internal volumes greater than about 20.0 liters would likely be useful for storage and transport of the gas/liquid mixture of nitric oxide in deionized and/or deoxygenated water and not for integration with a dispensing system. The dispensing volume or dosing volume of the gas/liquid mixture of nitric oxide in the deionized and/or deoxygenated water is preferably between about 3 ml to 100 ml, and more preferably about 10 ml. Lastly, the temperature range for the storage and transport of containment vessels of nitric oxide in deionized and/or deoxygenated water is between about −40° C. to 65° C., and more preferably below about 25° C. To increase the stability of nitric oxide in the deionized and/or deoxygenated water, the containment vessels may be refrigerated to temperatures below about 20° C.

Various embodiments of the dispensing system or the present containment vessel preferably include an electronic display capable of displaying information in digital and/or analog form and an electronic alarm capable of being actuated to signify the presence of an alarm state audibly, visibly, or both audibly and visibly during the use of the vessel and associated dispensing system. In such embodiments the system may also include an electronic control processor connected to the various sensors, the electronic display, and the electronic alarm.

The electronic control is preferably a microprocessor based controller configured to receive one or more signals from the plurality of sensors as well as user inputted data or transmitted data signifying vessel contents, location, user information, and/or other conditions for use of the containment vessel or dispensing system. The data and information are processed by the electronic control with selected parameters and information being concurrently displayed on the electronic display, including for example, the pressure within the vessel and the number of dispensing doses remaining in the vessel.

The electronic control is preferably configured to generate a signal actuating the electronic alarm when the volume of the gas/liquid mixture of nitric oxide in deionized and/or deoxygenated water in the vessel reaches a preset or predetermined value suggesting the vessel is in an empty or almost empty condition and the vessel needs to be replaced. Use of the dispensing device when the vessel is empty or near empty (e.g. fewer than a prescribed number of dispensing dosages remaining) may lead to inadequate disinfection of the surfaces to be treated. In applications using a gas/liquid mixture of nitric oxide in deionized and/or deoxygenated water vessel, an audible or visual alarm or indicator may also be activated when a dose is dispensed. Such dispensing alarm or indicator is useful because a user may not realize whether or not any solution was actually dispensed, as the dispensing system often aerosolizes the gas/liquid mixture prior to contact with the surfaces to be sanitized or disinfected. Thus, the dispensing alarm or indicator confirms to the user that the solution was dispensed.

The electronic control is further configured to generate a signal actuating the electronic alarm when the pressure of the gas/liquid mixture of nitric oxide in deionized and/or deoxygenated water in the vessel reaches a preset or predetermined low threshold. In order to ensure the dispensed solution likely has sufficient nitric oxide necessary to achieve the desired microbial kill, the pressure of the solution in the vessel needs to above a prescribed threshold pressure. If the pressure in the vessel is somehow reduced to a value at or below the low pressure threshold, there may be a leak in the system or the nitric oxide may come out of solution thus rendering the dispensed solution ineffective for sanitation and disinfection purposes. Use of a low pressure alarm identifies conditions when the dispensing device should not be used and the vessel or dispensing system should be replaced.

The electronic display can be configured to display either graphical information or numerical information, or both. Any useful information can be displayed, such as: the pressure of gas/liquid mixture in the containment vessel; the amount of gas/liquid mixture remaining in the containment vessel; the number of dispensing doses remaining until the amount of solution remaining in the containment vessel is low enough to reach a predetermined threshold value or to be completely exhausted from the containment vessel; the status (including alarm status) of the containment vessel, or other desired information. The format of the display can take the form of a symbol that comes on or flashes, an analog scale and/or a digital display, or other formats. The display can be activated so that different items of information appear together, or alternatingly (i.e. with one item appearing, then a second item, then the first again, and so on).

The disclosed containment vessel and dispensing system may also be fitted with additional sensors such as a chemical sensor, a temperature sensor, light sensor, accelerometer, magnetic field sensing, etc. to provide additional functionality for the device. A device incorporating a chemical sensor would be useful to detect impurities or presence of toxic gases and/or to assure the proper gas composition is delivered from the cylinder during use, particularly medical gas uses. For example, a built-in chemical sensor may be used to determine if effluents from the containment vessel or the dispensing device are safe and/or meet the required quality targets prior to any further use/dispensing. This is of critical importance in situations where toxic gases such as nitrogen dioxide may be present.

Other sensors that could be incorporated within the disclosed dispensing system and/or containment vessel may include the capability to sense proximity to a magnetic field of a given strength or the ability to detect temperature excursions outside of specific window and disable the device or emit an alarm if such temperature excursions or presence of magnetic fields pose a safety hazard. Another type of sensor suitable for integrating into the containment vessel might include an accelerometer to monitor/assure proper handling of the vessel or emit an alarm if the cylinder had been dropped or excessively mishandled during transport. Yet another sensor or detecting means that monitors the leak integrity of the containment vessel and/or dispensing system during an off state which and alerts the user that a leak may be present within the system. Such leak integrity sensors may be atmosphere monitoring sensors, chemical sensors, and/or pressure sensing techniques.

A particularly advantageous feature of the present dispensing system and/or containment vessel would be the ability to transmit and/or receive data to and/or from different external sources, such as a smartphone. For example, a global positioning system or GPS type chip may be integrated within the containment vessel and/or dispensing system to provide functionality regarding location or inventory tracking of the vessels. In addition, electronic transmission of the vessel contents to a data storage or central processing unit is contemplated for purposes of usage tracking, cylinder replacement planning, and other administrative or logistical functions. In medical applications, such communications would preferably be compliant or example with ISO/IEEE 11073-30300, “Health informatics—Point-of-care medical device communication—Part 30300, et seq. Such wireless communications incorporated within the device would preferably be configured with power saving features, so as to minimize power usage and preserve the battery life.

Certain embodiments of the present containment vessel and/or dispensing system preferable incorporate wireless or hard-wired communication features and, in particular, a data receiving capability which would allow use of externally supplied data to assist in the dispensing system. Wireless or hard wired communications can also be part of the security features of the present device. For example, operation of the device may be authorized through the use of RFID fobs, barcode scanning, or chip technology to identify an authorized user of the gas cylinder and device. The device may also incorporate anti-tampering features to identify situations where an unauthorized use of the device and release of the cylinder contents is attempted. An alternative arrangement would couple the present containment vessel and/or dispensing system to an external device which contains the communication and/or sensing functions and features described above.

The gas/liquid mixture of nitric oxide in deionized and/or deoxygenated water provided in embodiments disclosed herein may contain one or more type of additives such as preservatives, surfactants, solution stabilizers, anti-microbial agents, pH adjusters, or an agent that is capable of accelerating or inhibiting evaporation. As disclosed in U.S. Patent Application Publication No. 2014/0186211, certain additives that are preservatives may include butylated hydroyanisole (BHA), butylated hydroxytoluene (BHT), benzoic acid, ascorbic acid, methyl paraben, propyl paraben, tocopherols and mixtures thereof. Additives such as ammonia may be used to alkalinize the solution, or hydrochloric acid to acidify the solution.

As further disclosed in U.S. Patent Application Publication No. 2014/0186211, certain additives may include ethylene glycol or polyethylene glycol or other anti-microbial agents other than nitric oxide. Some of the other anti-microbial additives that may be used include but are not limited to halogenated aromatics, chlorinated hydrocarbons, organometallics, metallic salts, organic sulfur compounds, quaternary ammonium compounds, phenolics, triclosan, 3,4,4′-trichlorocarbanilide (triclocarban), 3,4,4′-trifluoromethyl-4,4′-dichlorocarbanilide (cloflucarban), 5-chloro-2-methyl-4-isothiazolin-3-one, iodopropynlbutylcarbamate, 8-hydroxyquinoline, 8-hydroxyquinoline citrate, 8-hydroxyquinoline sulfate, 4-chloro-3,5-xylenol(chloroxylenol), 2-bromo-2-nitropropane-1,3-diol, diazolidinyl urea, butylparaben, ethylparaben, methylparaben, methylchloroisothiazoline, methylisothiazoline, a mixture of 1,3-bis(hydroxymethyl)-5,5-dimethylhydantoin and 3-iodo-2-propynyl butyl carbamate, oxyquinoline, EDTA, tetrasodium EDTA, p-hydroxyl benzoic acid ester, alkyl pyridinum compounds, coco phosphatidyl PG-dimonium chloride, chlorhexidine gluconate, chlorhexidine digluconate, chlorhexidine acetate, chlorhexidine isethionate, chlorhexidine hydrochloride, benzalkonium chloride, benzethonium chloride, polyhexamethylene biguanide, and zinc salt, or mixtures thereof.

Although the present inventions have been discussed with reference to one or more preferred embodiments, as would occur to those skilled in the art that numerous changes and omissions can be made without departing from the spirit and scope of the present inventions as set forth in the appended claims.

What is claimed is:

1. A vessel for a gas/liquid mixture comprising nitric oxide in deionized and/or deoxygenated water, the vessel comprising:
a housing having a body section, a dispensing end, and an opposite end, the body section defining an interior chamber having one or more interior surfaces;
a moveable piston having a distal end, a proximal end, and one or more side surfaces disposed within the interior chamber such that the one or more side surfaces are sealingly engaged with the one or more interior surfaces of the interior chamber to define the first section of the interior chamber between the distal end of the piston and the dispensing end of the housing and to define the second section of the interior chamber between to the distal end of the piston and the opposite end of the housing;
wherein the dispensing end of the housing includes at least one dispensing port through which the gas/liquid mixture of nitric oxide in deionized and/or deoxygenated water exits the first section of the interior chamber as the moveable piston moves towards the dispensing end of the housing; and
wherein at least one of the one or more surfaces of the moveable piston remains in contact with a liquid surface of the gas/liquid mixture of nitric oxide in deionized and/or deoxygenated water in the first section of the interior chamber to prevent nitric oxide from coming out of the deionized and/or deoxygenated water.

2. The vessel of claim 1, wherein the gas/liquid mixture of nitric oxide in deionized and/or deoxygenated water is maintained at a prescribed pressure range above ambient pressure as the volume of the gas/liquid mixture within the first section of the interior chamber changes.

3. The vessel of claim 1, wherein the gas/liquid mixture of nitric oxide in deionized and/or deoxygenated water in the first section of the interior chamber is sealably isolated from any contaminants present in the second section of the interior chamber or from outside the housing.

4. The vessel of claim 1, wherein the moveable piston traverses the interior chamber toward the dispensing end of the housing in response to a mechanical force applied to or transferred to the moveable piston.

5. The vessel of claim 2, wherein the second section of the interior chamber is configured to receive a pneumatic or hydraulic fluid to maintain the gas/liquid mixture of nitric oxide in deionized and/or deoxygenated water in the first section of the interior chamber at the prescribed pressure range above ambient pressure.

6. The vessel of claim 2, wherein the second section of the interior chamber is configured to receive a pneumatic or hydraulic fluid to move the moveable piston and dispense the gas/liquid mixture of nitric oxide in deionized and/or deoxygenated water through the dispensing port.

7. The vessel of claim 1, wherein the housing, including components thereof and the moveable piston is constructed of materials selected from the group consisting of polyvinylchloride, aluminum, stainless steel, and fluoropolymer elastomers.

8. The vessel of claim 1, wherein one or more characteristics of the gas/liquid mixture of nitric oxide in deionized and/or deoxygenated water contained in the first section of the interior chamber are stored in a processor operatively coupled to the vessel.

9. The vessel of claim 8, further comprising a temperature sensor or a pressure sensor and wherein the sensed pressure or sensed temperature of the gas/liquid mixture of nitric oxide in deionized and/or deoxygenated water contained in the first section of the interior chamber are displayed on a gauge or a display attached to an exterior surface of the vessel.

10. The vessel of claim 8, wherein the volume of the gas/liquid mixture of nitric oxide in deionized and/or deoxygenated water contained in the first section of the interior chamber are displayed on a gauge or a display attached to an exterior surface of the vessel.

11. The vessel of claim 10, further comprising an alarm when the pressure of the gas/liquid mixture of nitric oxide in deionized and/or deoxygenated water contained in the first section of the interior chamber is outside a predetermined pressure range or below a pressure threshold.

12. The vessel of claim 10, further comprising an alarm when the temperature of the gas/liquid mixture of nitric oxide in deionized and/or deoxygenated water contained in the first section of the interior chamber is outside a predetermined temperature range or temperature threshold.

13. The vessel of claim 10, further comprising an alarm when the volume of the gas/liquid mixture of nitric oxide in deionized and/or deoxygenated water contained in the first section of the interior chamber is below a predetermined volume threshold.

14. The vessel of claim 8, wherein the processor is configured to store data about the gas/liquid mixture of nitric oxide in deionized and/or deoxygenated water contained in the first section of the interior chamber selected from the group consisting of characteristics of the gas/liquid mixture of nitric oxide in deionized and/or deoxygenated water in the first section of the interior chamber, temperature of the gas/liquid mixture of nitric oxide in deionized and/or deoxygenated water in the first section of the interior chamber, pressure of the gas/liquid mixture of nitric oxide in deionized and/or deoxygenated water in the first section of the interior chamber, volume of the gas/liquid mixture of nitric oxide in deionized and/or deoxygenated water in the first section of the interior chamber, any alarm condition, or combinations thereof.

15. The vessel of claim 14, wherein the processor is further configured to transmit some or all of the data stored in the processor to another device via a wireless or wired connection.

16. The vessel of claim 1, further comprising a filling port disposed on the housing and in fluidic communication with the first section of the interior chamber and configured to permit selective filling of the gas/liquid mixture of nitric oxide in deionized and/or deoxygenated water into the first section of the interior chamber.

17. The vessel of claim 1, further comprising a port disposed on the housing and in fluidic communication with the second section of the interior chamber and configured to permit selective filling of a pneumatic or hydraulic medium into the second section of the interior chamber and venting or release of the pneumatic or hydraulic medium from the second section of the interior chamber.

18. A dispensing system for a gas/liquid mixture of nitric oxide gas in deionized and/or deoxygenated water, the dispensing system comprising:
a container having a body section defining an interior chamber having one or more interior surfaces; a dispensing end; and an opposite end;
a moveable piston disposed within the interior chamber of the container, the moveable piston having a distal end, a proximal end, and one or more side surfaces, the moveable piston configured such that the one or more side surfaces are sealingly engaged with the one or more interior surfaces of the interior chamber of the container to define a first section of the interior chamber between to the distal end of the moveable piston and dispensing end of the container and a second section of the interior chamber between to the proximal end of the moveable piston and the opposite end of the container; and
a motive source in operative association with the container and the moveable piston and configured to cause the moveable piston to traverse the interior chamber of the container;
wherein the dispensing end of the container includes at least one port through which the gas/liquid mixture of nitric oxide in deionized and/or deoxygenated water is dispensed as the moveable piston traverses the interior chamber of the container toward the dispensing end of the container; and
wherein the distal end of the moveable piston remains in contact with a liquid surface of the gas/liquid mixture of nitric oxide in deionized and/or deoxygenated water in the first section of the interior chamber so as to prevent nitric oxide from coming out of the deionized and/or deoxygenated water.

19. The dispensing system of claim 18, wherein the gas/liquid mixture of nitric oxide in deionized and/or deoxygenated water is maintained at a prescribed pressure range above ambient pressure as the volume of the gas/liquid mixture within the first section of the interior chamber changes.

20. The dispensing system of claim 18, wherein the gas/liquid mixture of nitric oxide in deionized and/or deoxygenated water in the first section of the interior chamber is sealably isolated from any contaminants present in the second section of the interior chamber or from outside the container.

21. The dispensing system of claim 18, wherein the moveable piston traverses the interior chamber toward the dispensing end of the housing in response to a mechanical force applied to or transferred to the moveable piston.

22. The dispensing system of claim 19, further comprising a source of pneumatic or hydraulic fluid and wherein the second section of the interior chamber is configured to receive the pneumatic or hydraulic fluid to move the moveable piston and dispense the gas/liquid mixture of nitric oxide in deionized and/or deoxygenated water through the dispensing port.

23. The dispensing system of claim 19, further comprising a source of pneumatic or hydraulic fluid and wherein the second section of the interior chamber is configured to receive the pneumatic or hydraulic fluid to maintain the gas/liquid mixture of nitric oxide in deionized and/or deoxygenated water in the first section of the interior chamber at the prescribed pressure range above ambient pressure.

24. The dispensing system of claim 18, wherein the container, including components thereof and the moveable piston are constructed of materials selected from the group consisting of polyvinylchloride, aluminum, stainless steel, and fluoropolymer elastomers.

25. The dispensing system of claim 18, further comprising:
a processor operatively coupled to the container; and
a gauge or electronic display operatively coupled to the processor;
wherein one or more characteristics of the gas/liquid mixture of nitric oxide in deionized and/or deoxygenated water within the container are communicated or stored in the processor and displayed on the gauge or the electronic display.

26. The dispensing system of claim 25, wherein the volume of the gas/liquid mixture of nitric oxide in deionized and/or deoxygenated water contained in the container is displayed on the gauge or the electronic display.

27. The dispensing system of claim 26, further comprising an alarm configured to be activated when the volume of the gas/liquid mixture of nitric oxide in deionized and/or deoxygenated water in the container is below a predetermined volume threshold.

28. The dispensing system of claim 25, further comprising a temperature sensor and/or a pressure sensor and wherein the sensed pressure or sensed temperature of the gas/liquid mixture of nitric oxide in deionized and/or deoxygenated water in the container are displayed on the gauge or the electronic display.

29. The dispensing system of claim 28, further comprising an alarm configured to be activated when the pressure of the gas/liquid mixture of nitric oxide in deionized and/or deoxygenated water in the container is outside a predetermined pressure range or below a pressure threshold.

30. The dispensing system of claim 28, further comprising an alarm configured to be activated when the temperature of the gas/liquid mixture of nitric oxide in deionized and/or deoxygenated water in the container is outside a predetermined temperature range or temperature threshold.
